(19) United States
(12) Patent Application Publication
(10) Pub. No.: US 2025/0197392 A1
(43) Pub. Date: Jun. 19, 2025

(54) CRYSTALLINE FORMS OF AN INHIBITOR OF THE MENIN/MLL INTERACTION

(71) Applicant: JANSSEN PHARMACEUTICA NV, Beerse (BE)

(72) Inventors: Wei CAI, Shanghai (CN); Johannes Wilhelmus J. THURING, Antwerp (BE); Fabian HULPIA, Sint-Niklaas (BE); Xuedong DAI, Shanghai (CN); Ming LI, Shanghai (CN); Alicia Tee Fuay NG, Shanghai (CN); Nicolas Freddy Jacques Bruno DARVILLE, Linkhout (BE); Vineet PANDE, Vosselaar (BE)

(21) Appl. No.: 18/851,830

(22) PCT Filed: Apr. 7, 2023
(86) PCT No.: PCT/CN2023/086884

§ 371 Date: Sep. 27, 2024

(30) Foreign Application Priority Data
Apr. 8, 2022 (WO) PCT/CN2022/085680
May 30, 2022 (WO) PCT/CN2022/095901

(51) Int. Cl. C07D 471/04; A61K 31/4545; A61P 35/02

(52) U.S. Cl. CPC C07D 471/04; A61K 31/4545; A61P 35/02

(57) ABSTRACT

The present invention relates to crystalline forms of an inhibitor of menin/mixed lineage leukemia (MLL) protein-protein interaction. The present invention also relates to pharmaceutical compositions comprising crystalline forms of an inhibitor of menin/mixed lineage leukemia (MLL) protein-protein interaction. These crystalline forms and pharmaceutical compositions comprising said crystalline forms may be useful for treating diseases such as cancer.

### FIELD OF THE INVENTION

The present invention relates to crystalline forms of an inhibitor of menin/mixed lineage leukemia (MLL) protein-protein interaction. The present invention also relates to pharmaceutical compositions comprising crystalline forms of an inhibitor of menin/mixed lineage leukemia (MLL) protein-protein interaction. These crystalline forms and pharmaceutical compositions comprising said crystalline forms may be useful for treating diseases such as cancer.

### BACKGROUND OF THE INVENTION

Chromosomal rearrangements affecting the mixed lineage leukemia gene (MLL; MLL1; KMT2A) result in aggressive acute leukemias across all age groups and still represent mostly incurable diseases emphasizing the urgent need for novel therapeutic approaches. Acute leukemias harboring these chromosomal translocations of MLL represent as lymphoid, myeloid or biphenotypic disease and constitute 5 to 10% of acute leukemias in adults and approximately 70% in infants.

MLL is a histone methyltransferase that methylates histone H3 on lysine 4 (H3K4) and functions in multiprotein complexes. Use of inducible loss-of-function alleles of MLL1 demonstrated that MLL1 plays an essential role in sustaining hematopoietic stem cells (HSCs) and developing B cells although its histone methyltransferase activity is dispensable for hematopoiesis.

Fusion of MLL with more than 60 different partners has been reported to date and has been associated with leukemia formation/progression. Interestingly, the SET (Su(var)3-9, enhancer of zeste, and trithorax) domain of MLL is not retained in chimeric proteins but is replaced by the fusion partner. Recruitment of chromatin modifying enzymes like Dot1L and/or the pTEFb complex by the fusion partner leads to enhanced transcription and transcriptional elongation of MLL target genes including HOXA genes (e.g. HOXA9) and the HOX cofactor MEIS1 as the most prominent ones. Aberrant expression of these genes in turn blocks hematopoietic differentiation and enhances proliferation.

Menin which is encoded by the Multiple Endocrine Neoplasia type 1 (MEN1) gene is expressed ubiquitously and is predominantly localized in the nucleus. It has been shown to interact with numerous proteins and is, therefore, involved in a variety of cellular processes. The best understood function of menin is its role as an oncogenic cofactor of MLL fusion proteins. Menin interacts with two motifs within the N-terminal fragment of MLL that is retained in all fusion proteins, MBM1 (menin-binding motif 1) and MBM2. Menin/MLL interaction leads to the formation of a new interaction surface for lens epithelium-derived growth factor (LEDGF). Although MLL directly binds to LEDGF, menin is obligatory for the stable interaction between MLL and LEDGF and the gene specific chromatin recruitment of the MLL complex via the PWWP domain of LEDGF. Furthermore, numerous genetic studies have shown that menin is strictly required for oncogenic transformation by MLL fusion proteins suggesting the menin/MLL interaction as an attractive therapeutic target. For example, conditional deletion of MEN1 prevents leukomogenesis in bone marrow progenitor cells ectopically expressing MLL fusions. Similarly, genetic disruption of menin/MLL fusion interaction by loss-of-function mutations abrogates the oncogenic properties of the MLL fusion proteins, blocks the development of leukemia in vivo and releases the differentiation block of MLL-transformed leukemic blasts. These studies also showed that menin is required for the maintenance of HOX gene expression by MLL fusion proteins. In addition, small molecule inhibitors of menin/MLL interaction have been developed suggesting druggability of this protein/protein interaction and have also demonstrated efficacy in preclinical models of AML. Together with the observation that menin is not a requisite cofactor of MLL1 during normal hematopoiesis, these data validate the disruption of menin/MLL interaction as a promising new therapeutic approach for the treatment of MLL-rearranged leukemia and other cancers with an active HOX/MEIS1 gene signature. For example, an internal partial tandem duplication (PTD) within the 5′ region of the MLL gene represents another major aberration that is found predominantly in de novo and secondary AML as well as myeloid dysplasia syndromes. Although the molecular mechanism and the biological function of MLL-PTD is not well understood, new therapeutic targeting strategies affecting the menin/MLL interaction might also prove effective in the treatment of MLL-PTD-related leukemias. Furthermore, castration-resistant prostate cancer has been shown to be dependent on the menin/MLL interaction.

MLL protein is also known as Histone-lysine N-methyltransferase 2A (KMT2A) protein in the scientific field (UniProt Accession #Q03164).

WO2022/253167 relates to menin/MLL protein/protein interaction inhibitors.

### DESCRIPTION OF THE INVENTION

The present invention is directed to crystalline forms of 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide having the following structure:

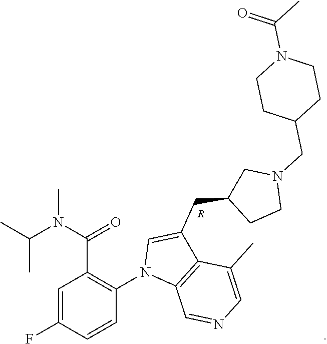

In an embodiment, the crystalline form of 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide is a crystalline free base Form.

In an embodiment, the crystalline form of 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide is a crystalline HCl salt Form; in particular a crystalline mono HCl salt variable hydrate; more in particular a crystalline mono HCl salt trihydrate.

The present invention also relates to a pharmaceutical composition comprising a therapeutically effective amount of a crystalline form of 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide.

The present invention also relates to a pharmaceutical composition comprising a therapeutically effective amount of a crystalline form of 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide, and a pharmaceutically acceptable carrier or excipient.

Additionally, the invention relates to a crystalline form of 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide for use as a medicament, and to a crystalline form of 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide for use in the treatment or in the prevention of cancer, including but not limited to leukemia, myelodysplastic syndrome (MDS), and myeloproliferative neoplasms (MPN); and diabetes.

The present invention also relates to a pharmaceutical composition comprising a therapeutically effective amount of a crystalline form of 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide, for use in the treatment or in the prevention of cancer, including but not limited to leukemia, myelodysplastic syndrome (MDS), and myeloproliferative neoplasms (MPN); and diabetes.

The present invention also relates to a pharmaceutical composition comprising a therapeutically effective amount of a crystalline form of 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide, and a pharmaceutically acceptable carrier or excipient, for use in the treatment or in the prevention of cancer, including but not limited to leukemia, myelodysplastic syndrome (MDS), and myeloproliferative neoplasms (MPN); and diabetes. In a particular embodiment, the invention relates to a crystalline form of 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide, for use in the treatment or in the prevention of cancer.

In a specific embodiment said cancer is selected from leukemias, lymphomas, myelomas or solid tumor cancers (e.g. prostate cancer, lung cancer, breast cancer, pancreatic cancer, colon cancer, liver cancer, melanoma and glioblastoma, etc.). In some embodiments, the leukemias include acute leukemias, chronic leukemias, myeloid leukemias, myelogeneous leukemias, lymphoblastic leukemias, lymphocytic leukemias, Acute myelogeneous leukemias (AML), Chronic myelogenous leukemias (CML), Acute lymphoblastic leukemias (ALL), Chronic lymphocytic leukemias (CLL), T cell prolymphocytic leukemias (T-PLL), Large granular lymphocytic leukemia, Hairy cell leukemia (HCL), MLL-rearranged leukemias, MLL-PTD leukemias, MLL amplified leukemias, MLL-positive leukemias, leukemias exhibiting HOX/MEIS1 gene expression signatures etc.

In particular, compounds according to the present invention and the pharmaceutical compositions thereof may be useful in the treatment or prevention of leukemias, in particular nucleophosmin (NPM1)-mutated leukemias, e.g. NPM1c.

In an embodiment, compounds according to the present invention, may have improved metabolic stability properties.

In an embodiment, compounds according to the present invention, may have extended in vivo half-life (T1/2).

In an embodiment, compounds according to the present invention, may have improved oral bioavailability.

In an embodiment, compounds according to the present invention, may reduce tumor growth e.g., tumours harbouring MLL (KMT2A) gene rearrangements/alterations and/or NPM1 mutations.

In an embodiment, compounds according to the present invention, may have improved PD properties in vivo during a prolonged period of time, e.g. inhibition of target gene expression such as MEIS1 and upregulation of differentiation marker over a period of at least 16 hours.

In an embodiment, compounds of Formula (I) and the pharmaceutically acceptable salts, and the solvates thereof, may have an improved safety profile (e.g. reduced hERG inhibition; improved cardiovascular safety).

In an embodiment, compounds according to the present invention, may be suitable for Q.D. dosing (once daily).

The invention also relates to the use of compounds according to the present invention, in combination with an additional pharmaceutical agent for use in the treatment or prevention of cancer, including but not limited to leukemia, myelodysplastic syndrome (MDS), and myeloproliferative neoplasms (MPN); and diabetes.

Furthermore, the invention relates to a process for preparing a pharmaceutical composition according to the invention, characterized in that a pharmaceutically acceptable carrier is intimately mixed with a therapeutically effective amount of a compound according to the present invention.

The invention also relates to a product comprising a compound according to the present invention, and an additional pharmaceutical agent, as a combined preparation for simultaneous, separate or sequential use in the treatment or prevention of cancer, including but not limited to leukemia, myelodysplastic syndrome (MDS), and myeloproliferative neoplasms (MPN); and diabetes.

Additionally, the invention relates to a method of treating or preventing a cell proliferative disease in a warm-blooded animal which comprises administering to the animal an effective amount of a compound according to the present invention, as defined herein, or a pharmaceutical composition or combination as defined herein.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific disclosure of the drawings. In the drawings:

FIG. 1 is an X-ray powder diffraction (XRPD) pattern of 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide as a crystalline free base Form.

FIG. 2 is an X-ray powder diffraction (XRPD) pattern of 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide as a crystalline HCl salt Form.

FIG. 3 is a Dynamic vapor sorption (DVS) isotherm plot of 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide as a crystalline HCl salt Form.

FIG. 4 is a Dynamic vapor sorption (DVS) change in mass plot of 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide as a crystalline HCl salt Form.

### DETAILED DESCRIPTION OF THE INVENTION

The term “compound(s) of the (present) invention” or “compound(s) according to the (present) invention” as used herein, is meant to include crystalline forms of 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide, such as for example a crystalline HCl salt Form, and a crystalline free base Form.

The term “subject” as used herein, refers to an animal, preferably a mammal (e.g. cat, dog, primate or human), more preferably a human, who is or has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medicinal doctor or other clinician, which includes alleviation or reversal of the symptoms of the disease or disorder being treated.

The term “composition” is intended to encompass a product comprising the specified ingredients in the specified amounts, as well as any product which results, directly or indirectly, from combinations of the specified ingredients in the specified amounts.

The term “treatment”, as used herein, is intended to refer to all processes wherein there may be a slowing, interrupting, arresting or stopping of the progression of a disease, but does not necessarily indicate a total elimination of all symptoms.

It will be clear for a skilled person that the present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature).

All isotopes and isotopic mixtures of any particular atom or element as specified herein are contemplated within the scope of the compounds of the invention, either naturally occurring or synthetically produced, either with natural abundance or in an isotopically enriched form.

In an embodiment, the compound of the present invention is 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide as a crystalline free base Form.

In an embodiment, the compound of the present invention is 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide as a crystalline HCl salt Form; in particular a crystalline mono HCl salt variable hydrate of 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide; more in particular a crystalline mono HCl salt trihydrate of 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide.

The present invention also relates to a pharmaceutical composition comprising a compound of the present invention.

The present invention also relates to a pharmaceutical composition comprising a therapeutically effective amount of a compound of the present invention and a pharmaceutically acceptable carrier or excipient.

### Experimental Part

Several methods for preparing the compounds of this invention are illustrated in the following examples. Unless otherwise noted, all starting materials were obtained from commercial suppliers and used without further purification, or alternatively can be synthesized by a skilled person by using well-known methods.

| Abbreviation | Meaning |
|---|---|
| CH₃COONH₄ | ammonium acetate |
| sat. or Sat. | saturated |
| ° C. | degree Celsius |
| AcOH or CH₃COOH | acetic acid |
| aq. | aqueous |
| atm | atmosphere |
| Boc or boc | tert-butyloxycarbonyl |
| BOC-anhydride | di-tert-butyl dicarbonate |
| BPin or PinB | 4,4,5,5-tetramethyl-1,3,2-dioxaborolane |
| Celite | diatomaceous earth |
| CO₂ | carbon dioxide |
| Cs₂CO₃ | cesium carbonate |
| CPME | cyclopentyl methylether |
| DCM or CH2Cl2 | dichloromethane |
| DEA | diethanolamine |
| DEE | diethyl ether |
| DIEA or DIPEA | N-ethyl-N-(propan-2-yl)propan-2-amine |
| DMA | N,N-dimethylacetamide |
| DME | 1,2-dimethoxyethane |
| DMF | N,N-dimethylformamide |
| DMSO | (methanesulfinyl)methane |
| DSC | differential scanning calorimetry |
| EDCI or EDCI•HCl | 3-{[(ethylimino)methylidene]amino}-N,N- |
| | dimethylpropan-1-amine |
| ee | enantiomeric excess |
| ESI | electrospray ionization |
| EtOAc or EA | ethyl acetate |
| EtOH | ethanol |
| FA | formic acid |
| FCC | flash column chromatography |
| h or hr | hour(s) |
| HATU | 1-[bis(dimethylamino)methylene]-1H-1,2,3- |
| | triazolo[4,5-b]pyridinium |
| | 3-oxide hexafluorophosphate |
| HCl | hydrochloric acid |
| Hex | hexane |
| HOBt | 1-hydroxybenzotriazole |
| i-PrNH₂ or iPrNH | isopropyl amine |
| i-PrOH, iPrOH or IPA | isopropyl alcohol |
| IPAC | isopropyl acetate |
| K₂CO₃ | potassium carbonate |
| KOAc | potassium acetate |
| LCMS | liquid chromatography-mass spectrometry |
| LiAlH₄ | lithium aluminium hydride |
| Li-HMDS or LiHMDS | lithium bis(trimethylsilyl)amide |
| M or N | mol/L |
| MeCN or CH₃CN | acetonitrile |
| MeI | iodomethane |
| MeOH | methanol |
| mg | milligram |
| min | minute(s) |
| mL | milliliter |
| mmol | millimole |
| MS | mass spectrometry |
| N₂ | nitrogen |
| Na₂CO₃ | sodium carbonate |
| Na₂SO₄ | sodium sulfate |
| NaBH(OAc)₃ | sodium triacetoxyborohydride |
| NaCNBH₃ | sodium cyanoborohydride |
| Na₄EDTA | Ethylenediaminetetraacetic acid tetrasodium salt |
| NaHCO₃ | sodium hydrogencarbonate |
| NaOAc | sodium acetate |
| NaOH | sodium hydroxide |
| NH₃ | ammonia |
| NH₃•H₂O or NH₄OH | ammonium hydroxide |
| NH₄HCO₃ | ammonium hydrogencarbonate |
| n-BuLi | n-butyllithium |
| NBS | N-Bromosuccinimide |
| NH₄Cl | ammonium chloride |
| NMP | 1-methyl-2-pyrrolidinone |
| NMR | nuclear magnetic resonance |
| Pd(dppf)Cl₂ | [1,1′- |
| | bis(diphenylphosphino)ferrocene]di- |
| | chloropalladium(II) |
| Pd(PPh₃)₄ | tetrakis(triphenylphosphine)palladium(0) |
| Pd/C | palladium on carbon |
| PE | petroleum ether |
| Prep. HPLC | preparative high-performance |
| | liquid chromatography |
| Prep. SFC | preparative supercritical |
| | fluid chromatography |
| Prep. TLC | preparative thin-layer chromatography |
| Rf | retention factor |
| RP | Reverse(d) phase |
| rt, r.t. or RT | room temperature |
| RuPhos Pd G4/ | Palladium, [[2′,6′-bis(1- |
| 4^{th} generation | methylethoxy)[1,1′-biphenyl]-2- |
| RuPhos Pd | yl]dicyclohexylphosphine-κP](methanesulfonato- |
| precatalyst | κO)[2′-(methylamino-κN)[1,1′- |
| | biphenyl]-2-yl-κC]-, (SP-4-3)- (ACI) |
| | CAS 1599466-85-9 |
| sat. | saturated |
| SFC | supercritical fluid chromatography |
| t-butyl | tert-butyl |
| t-BuOK | potassium tert-butoxide |
| T₃P | propylphosphonic anhydride |
| TEA or Et₃N | triethylamine |
| Temp | temperature |
| TFA | trifluo acetic acid |
| THF | tetrahydrofuran |
| TLC | thin-layer chromatography |
| Tₒₙₛₑₜ | Temperature at which melting onset occurs |
| | (measure by DSC) |
| Ts | tosyl |
| UV | ultraviolet |
| v/v | volume to volume |
| w/v | weight to volume |
| w/w | weight to weight |
| ZnCl₂ | zinc chloride |

Compounds or intermediates isolated as a salt form, may be integer stoichiometric i.e. mono- or di-salts, or of intermediate stoichiometry. When an intermediate or compound in the experimental part below is indicated as ‘HCl salt’ without indication of the number of equivalents of HCl, this means that the number of equivalents of HCl was not determined.

The stereochemical configuration for centers in some compounds/intermediates may be designated “R” or “S” when the mixture(s) was separated and absolute stereochemistry was known, or when only one enantiomer was obtained and absolute stereochemistry was known; for some intermediates, the stereochemical configuration at indicated centers has been designated as “*R” or “*S” when the absolute stereochemistry is undetermined (even if the bonds are drawn stereo specifically) although the intermediate itself has been isolated as a single stereoisomer and is enantiomerically pure. In case a compound designated as “*R” is converted into another compound, the “*R” indication of the resulting compound is derived from its starting material.

For example, it will be clear that intermediate 18

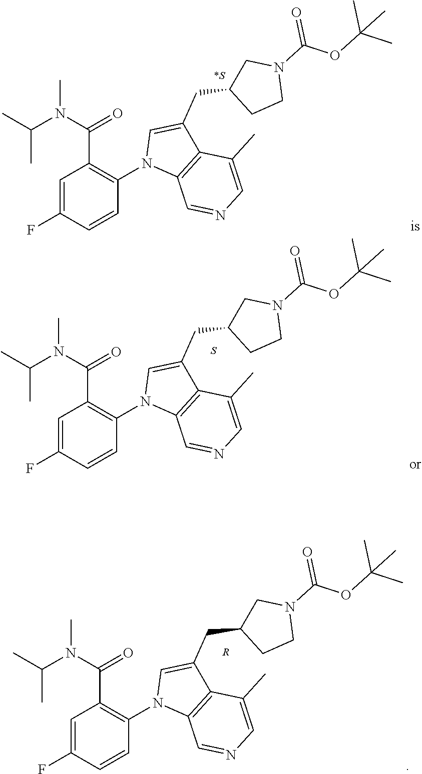

A skilled person will realize that, even where not mentioned explicitly in the experimental protocols below, typically after a column chromatography purification, the desired fractions were collected and the solvent was evaporated.

In case no stereochemistry is indicated, this means it is a mixture of stereoisomers or undetermined stereochemistry, unless otherwise is indicated or is clear from the context.

When a stereocenter is indicated with ‘RS’ this means that a racemic mixture was obtained at the indicated centre, unless otherwise indicated.

A double bond indicated with EZ means the compound/intermediate was obtained as a mixture of E and Z isomers.

### Preparation of Intermediates and Compounds

For intermediates that were used in a next reaction step as a crude or as a partially purified intermediate, in some cases no mol amounts are mentioned for such intermediate in the next reaction step or alternatively estimated mol amounts or theoretical mol amounts for such intermediate in the next reaction step are indicated in the reaction protocols described below.

### Preparation of Intermediate 1:

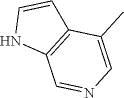

To a solution of 4-bromo-1H-pyrrolo[2,3-c]pyridine (2 g, 95% purity, 9.64 mmol) in 1,4-dioxane (30 mL) and water (4 mL) was added 2,4,6-trimethyl-1,3,5,2,4,6-trioxatriborinane (7.26 g, 50% in THF, 28.9 mmol) and potassium carbonate (4.0 g, 28.9 mmol). The suspension was degassed and exchanged with N₂ twice. [1,1′-bis(diphenylphosphino)ferrocene]dichloropalladium(II) (706 mg, 0.964 mmol) was added into the reaction mixture. The reaction mixture was heated up to 100° C. and stirred at this temperature overnight. After cooled down to r.t., the reaction mixture was filtered and the filtrate was concentrated. The resulting residue was purified by silica gel column chromatography eluting with ethyl acetate in petroleum ether from 0% to 80% to give intermediate 1 (1.01 g, 95% purity, 75.3% yield).

Alternatively, intermediate 1 can also be prepared with the following procedure:

Into a 20 L 4-necked round-bottom flask were added 4-bromo-1H-pyrrolo[2,3-c]pyridine (1330 g, 6750 mmol, 1.00 equiv), Pd(dppf)Cl₂ (493.9 g, 675 mmol, 0.10 equiv), K₂CO₃ (2798.69 g, 20250.21 mmol, 3.00 equiv), 1,4-dioxane (13 L), H₂O (2 L) and 2,4,6-trimethyl-1,3,5,2,4,6-trioxatriborinane (2542.01 g, 20250.21 mmol, 3.00 equiv) at room temperature. The resulting mixture was stirred for overnight at 100° C. The mixture was allowed to cool down to room temperature. The resulting mixture was concentrated under reduced pressure.

The resulting mixture was diluted with water (15 L). The aqueous layer was extracted with EtOAc (3×10 L) and the organic layer was washed with water (2×5 L). The resulting liquid was dried with Na₂SO₄, filtered and concentrated under vacuum. The residue was purified by silica gel column chromatography, eluting with with 10% methanol in dichloromethane to afford intermediate 1 (640 g, yield: 72%) as a grey solid.

### Preparation of Intermediate 2:

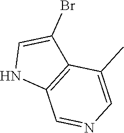

At 0° C., to a solution of intermediate 1 (918 mg, 95% purity, 6.6 mmol) in DMF (60 mL) was added a solution of N-bromosuccinimide (1.17 g, 6.6 mmol) in DMF (10 mL) dropwise. The reaction mixture was stirred at this temperature for 30 minutes. The reaction mixture was quenched with water and extracted with ethyl acetate (50 mL) twice. The organic layer was washed with brine (25 mL), dried over sodium sulfate, filtered and concentrated to afford the crude product, which was purified by silica gel column chromatography eluting with ethyl acetate in petroleum from 0% to 60% to give intermediate 2 (1.14 g, 97.1% purity, 79.5% yield) as a white solid.

Alternatively, intermediate 2 can also be prepared with the following procedure:

Into a 10 L 4-necked round-bottom flask were added intermediate 1 (640 g, 4842.39 mmol, 1.00 equiv) and DMF (5.00 L) at room temperature. To the above mixture was added NBS (861.87 g, 4842.40 mmol, 1.00 equiv) in portions over 1 h at room temperature. The resulting mixture was stirred for additional 30 min at room temperature. The reaction was quenched by the addition of aqueous solution of Na₂S₂O₃ (10 L, 10% (w/v)) at room temperature. The aqueous layer was extracted with EtOAc (3×5 L) and the organic layer was washed with brine (1×5 L). The resulting liquid was dried with Na₂SO₄ and concentrated. The residue was purified by silica gel column chromatography, eluting with 20% ethyl acetate in petroleum ether to afford intermediate 2 (800 g, yield: 78%) as a grey solid.

### Preparation of Intermediate 4:

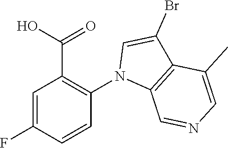 [SMILES: Cc1cncc2c1c(Br)cn2-c1ccc(F)cc1C(=O)O]

To a solution of intermediate 2 (1.14 g, 97.1% purity, 5.24 mmol) in DMF (80 mL) were added 5-fluoro-2-iodobenzoic acid (1.40 mg, 5.24 mmol), copper powder (333 mg, 5.24 mmol) and potassium carbonate (2.18 g, 15.7 mmol). The reaction mixture was heated up to 100° C. and stirred at this temperature overnight. After the mixture was cooled down to r.t., the reaction mixture was concentrated and the resulting residue was acidified with HCl (1 N) to pH=−3. The resulting mixture was filtered and the filter cake was washed with water twice. The filter cake was dried under vacuum to give crude intermediate 4 (1.8 g, 91% purity, 89.4% yield) as a yellow solid.

Alternatively, intermediate 4 can also be prepared with the following procedure:

Into a 10 L 4-necked round-bottom flask were added intermediate 2 (560 g, 2653.24 mmol, 1.00 equiv), Cu (252.91 g, 3979.87 mmol, 1.50 equiv), K₂CO₃ (1100.08 g, 7959.74 mmol, 3.00 equiv) and 5-fluoro-2-iodobenzoic acid (705.79 g, 2653.24 mmol, 1.00 equiv) in DMF (6.00 L) at room temperature. The resulting mixture was stirred for additional 2 h at 100° C. under nitrogen atmosphere. The resulting mixture was filtered, the filter cake was washed with DMF (1×5 L) and the filtrate was concentrated under reduced pressure. The resulting mixture was diluted with water (8 L). The mixture was acidified to pH 3 with aqueous HCl (conc.). The precipitated solids were collected by filtration and washed with water (3×3 L). The resulting solid was dried under vacuum to afford intermediate 4 (1300 g, crude) as a grey solid.

Intermediate 110 was synthesized by an analogous method starting from intermediate 1.

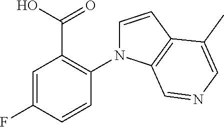 [SMILES: Cc1cncc2c1ccn2-c1ccc(F)cc1C(=O)O]

### Preparation of Intermediate 6:

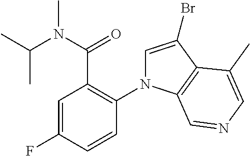 [SMILES: Cc1cncc2c1c(Br)cn2-c1ccc(F)cc1C(=O)N(C)C(C)C]

At 0° C., to a solution of intermediate 4 (1.8 g, 91% purity, 4.69 mmol) in DMF (50 mL) was added HATU (4.46 g, 11.7 mmol), N,N-diisopropylethylamine (3.03 g, 23.5 mmol) and N-methylpropan-2-amine (858 mg, 11.7 mmol). After addition, the mixture was stirred at room temperature overnight. The reaction mixture was concentrated and the resulting residue was purified by silica gel column chromatography eluted with methanol in dichloromethane from 0% to 5% to give intermediate 6 (2.0 g, 93% purity, 98.1% yield) as a yellow oil.

Alternatively, intermediate 6 can also be prepared with the following procedure:

Into a 20 L 4-necked round-bottom flask were added intermediate 4 (920 g, 2634.90 mmol, 1.00 equiv, same as 1300 g crude), DMF (7.5 L), HATU (1102.06 g, 2898.39 mmol, 1.10 equiv) and DIEA (1021.63 g, 7904.70 mmol, 3.00 equiv) at room temperature. The resulting mixture was stirred for additional 30 min at room temperature. To the above mixture was added N-methylpropan-2-amine (211.99 g, 2898.39 mmol, 1.10 equiv) dropwise over 10 min at 0° C. The resulting mixture was stirred overnight at room temperature. The reaction was quenched by the addition of water (20 L) at room temperature. The aqueous layer was extracted with EtOAc (3×7 L) and the organic layer was washed with water (3×5 L). The resulting liquid was dried with Na₂SO₄, filtered and concentrated under reduced pressure. The residue was purified by silica gel column chromatography, eluting with 50% ethyl acetate in petroleum ether (1:1) to afford intermediate 6 (700 g, yield: 66%) as a light yellow solid.

Intermediate 111 was synthesized by an analogous method starting from intermediate 110.

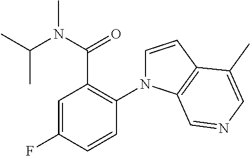 [SMILES: Cc1cncc2c1ccn2-c1ccc(F)cc1C(=O)N(C)C(C)C]

### Alternative Approach for the Preparation of Intermediate 6

Intermediate 111 (1.3 g, 4.0 mmol) was dissolved in MeCN (40 mL). Next, CuBr₂ (2.7 g, 12 mmol) was added, and the mixture was stirred at room temperature for 5 h. Next, 7N NH₃/MeOH (20 mL) was added. The reaction mixture was stirred vigorously for ˜30 min. Then, water (40 mL) and isopropyl acetate were added. The layers were separated, and the water layer was extracted twice with isopropyl acetate. The organic layers were combined, washed with brine, dried over Na₂SO₄, filtered and evaporated to dryness. The residue was purified by silica gel column chromatography eluting with methanol in dichloromethane from 0% to 3% to provide intermediate 6 (1.2 g, yield 72%) as an orange oil.

### Preparation of Intermediate 9:

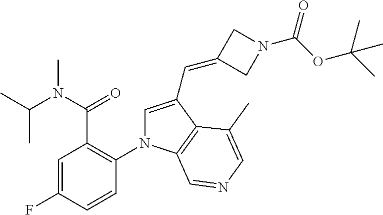 [SMILES: Cc1cncc2c1c(C=C1CN(C(=O)OC(C)(C)C)C1)cn2-c1ccc(F)cc1C(=O)N(C)C(C)C]

To a mixture of intermediate 6 (4 g, 4.312 mmol), tert-butyl 3-((4,4,5,5-tetramethyl-1,3,2-dioxaborolan-2-yl)methylene)azetidine-1-carboxylate (2.92 g, 9.9 mmol) and potassium carbonate (2.7 g, 19.7 mmol) in 1,4-dioxane (70 mL) and water (23 mL) was added Pd(dppf)Cl₂ (724 mg, 0.99 mmol). The mixture was degassed under nitrogen atmosphere three times and the reaction was stirred at 100° C. under nitrogen atmosphere for 16 h. After the mixture was cooled down to RT, the reaction mixture was diluted with H₂O and extracted with EtOAc. The combined organic phase was washed with brine, dried over Na₂SO₄, filtered and concentrated under vacuum. The residue was purified by silica gel column chromatography eluting with 90% ethyl acetate in petroleum ether to give intermediate 9 (1.8 g, 45.7% purity, 38.7% yield) as a yellow solid.

### Preparation of Intermediate 10:

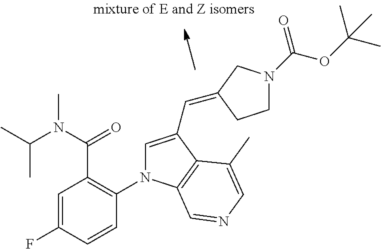 [SMILES: Cc1cncc2c1c(/C=C1\CCN(C(=O)OC(C)(C)C)C1)cn2-c1ccc(F)cc1C(=O)N(C)C(C)C]

A mixture intermediate 6 (12.0 g, 29.8 mmol), tert-butyl 3-((4,4,5,5-tetramethyl-1,3,2-dioxaborolan-2-yl)methylene)pyrrolidine-1-carboxylate (9.2 g, 29.8 mmol) and potassium carbonate (12.3 g, 89.1 mmol) in 1,4-dioxane (120 mL) and water (20 mL) was degassed and exchanged with N₂ twice. Pd(dppf)Cl₂ (2.16 g, 2.95 mmol) was added and the reaction mixture was heated up to 100° C. and stirred at this temperature overnight. After the reaction mixture was cooled down to r.t., the resulting mixture was concentrated and the residue was purified by silica gel column chromatography eluting with ethyl acetate in petroleum ether from 0% to 80% to give intermediate 10 (12.0 g, 79.4% yield) as a yellow oil.

### Preparation of Intermediate 15:

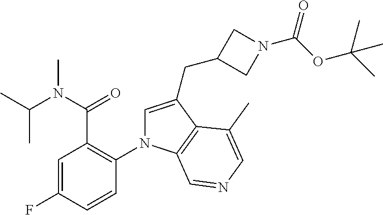 [SMILES: Cc1cncc2c1c(CC1CN(C(=O)OC(C)(C)C)C1)cn2-c1ccc(F)cc1C(=O)N(C)C(C)C]

A mixture of intermediate 9 (6.0 g, 12.2 mmol) in methanol (100 mL) was degassed under nitrogen atmosphere three times. 10 w/w % palladium on charcoal (3 g) was added and the mixture was degassed under hydrogen atmosphere three times. The mixture was stirred at r.t. under hydrogen atmosphere (balloon) for 16 h. The mixture was filtered and the filtrate was concentrated and purified by silica gel column chromatography eluting with 50% ethyl acetate in petroleum ether to give intermediate 15 (5.2 g, 97% purity, 83.7% yield) as a yellow solid.

### Preparation of Intermediate 16, 17 & 18:

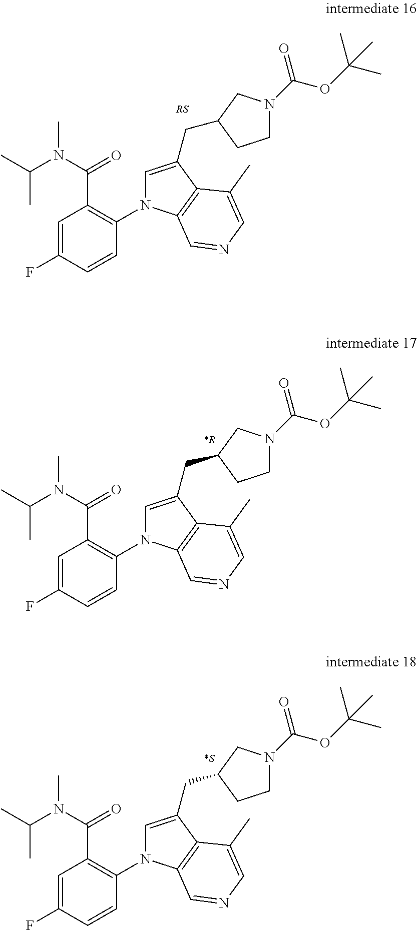

To a solution of intermediate 10 (2.5 g, 93% purity, 4.59 mmol) in methanol (40 mL) was added 10 w/w % palladium on charcoal (1 g) under N₂. The suspension was degassed under vacuum and purged with H₂ several times. The reaction mixture was heated up to 30° C. and stirred at this temperature overnight. After the reaction was cooled down to r.t., the reaction mixture was filtered and the filtrate was concentrated and purified by silica gel column chromatography eluted with methanol in dichloromethane from 0% to 5% to give intermediate 16 (2.5 g, 93% purity, 99.6% yield) as a yellow oil.

Intermediate 16 (8 g, 95% purity, 14.9 mmol) was separated by chiral IG-SFC (separation condition: Column: IG; Mobile Phase: CO₂-IPA: 65:35, at 60 mL/min; Temp: 40° C.; Wavelength: 214 nm) to afford intermediate 17 (first fraction, 3.29 g, 98% purity, 42.4% yield) as a yellow oil and intermediate 18 (second fraction, 3.36 g, 98% purity, 43.3% yield) as a yellow solid.

Chiral SFC method 2 was employed to match the stereochemistry of intermediate 18 and intermediate 201, retention time=5.97-6.10 min.

### Preparation of Intermediate 25

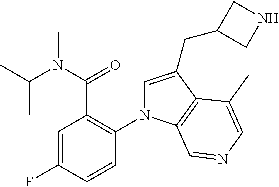 [SMILES: Cc1cncc2c1c(CC1CNC1)cn2-c1ccc(F)cc1C(=O)N(C)C(C)C]

To a cooled (ice bath) solution of intermediate 15 (1.1 g, 2.2 mmol) in dichloromethane (14 mL) was added dropwise TFA (7 mL). Then, the mixture was stirred at r.t. for 2 h. The solvent was removed by evaporation and the residue was dissolved in DCM, the pH was adjusted to 8-9 with saturated sodium carbonate aqueous solution, and extracted with DCM. The organic phase was dried over Na₂SO₄ and concentrated under vacuum to give intermediate 25 (680 mg, 72% yield) as a white solid.

### Intermediate 27 was Synthesized by an Analogous Method as Described for Intermediate 25

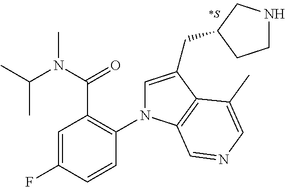 [SMILES: Cc1cncc2c1c(C[C@H]1CCNC1)cn2-c1ccc(F)cc1C(=O)N(C)C(C)C]

### Preparation of Intermediate 201—Method A:

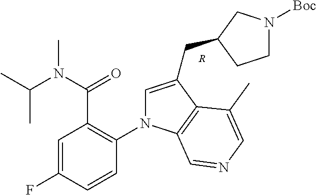

Into a 2 L 4-necked round-bottom flask were added THF (345 mL) and Zn (120.87 g, 1847.90 mmol, 5.00 equiv) at 30° C. under a nitrogen atmosphere. A solution of TMSCl (8.03 g, 73.91 mmol, 0.2 equiv) and 1-bromo-2-chloroethane (10.60 g, 73.91 mmol, 0.20 equiv) in THF (230 mL) were added into above round-bottom flask with a Lead Fluid-BT100F peristaltic pump (rate: 10 mL/min) under a nitrogen atmosphere. The resulting mixture was stirred for additional 40 min at 30° C. Next, a Lead Fluid-BT100F peristaltic pump was used to remove the solvent in above RBF quickly, and then fresh THF (575 mL) was re-charged under a nitrogen atmosphere. The mixture was heated to 60° C. Next, a solution of tert-butyl (3R)-3-(iodomethyl)pyrrolidine-1-carboxylate (115 g, 369.58 mmol, 1.00 equiv) in THF (575 mL) was added into above RBF with a Lead Fluid-BT100F peristaltic pump (rate: 15.0 mL/min) under a nitrogen atmosphere (temperature rises to 60-65° C.). The solution was stirred at 60° C. for an additional 1 h. The mixture was then cooled to 30° C. and allowed to stand for 1 h. The solution of {[(3R)-1-(tert-butoxycarbonyl)pyrrolidin-3-yl]methyl}(iodo)zinc was used directly in the next step. The concentration of the product was about 0.37 moL/L in THF.

Into a 2 L 4-necked round-bottom flask were added intermediate 6 (105 g, 259.71 mmol, 1.00 equiv) and THF (500 mL) at 30° C. under nitrogen atmosphere. To the stirred solution was added the 4th Generation RuPhos Pd precatalyst (5.65 g, 6.49 mmol, 0.025 equiv) under nitrogen atmosphere. Next, the solution of {[(3R)-1-(tert-butoxycarbonyl)pyrrolidin-3-yl]methyl}(iodo)zinc was added with a Lead Fluid-BT100F peristaltic pump into the 2 L 4-need RBF quickly under a nitrogen atmosphere (the excess zinc dust was not transferred). The resulting mixture was stirred for an additional 16 h at 50° C. The reaction was repeated 6 times in parallel. The reaction was quenched by the addition of aqueous sat. NH₄Cl solution (12 L). The aqueous layer was extracted with EtOAc (3×6 L), the organic layer was washed with water (2×3 L) and brine (1×3 L). The resulting mixture was dried with Na₂SO₄ and concentrated under reduced pressure. The crude product as a black oil (1100 g, crude) was used directly into the next step (preparation of intermediate 202)

### Alternatively, the Procedure Described Below can be Employed for the Preparation of Intermediate 201—Method B

A column (1.5 cm×15 cm) was stoppered with cotton wool and filled with granular zinc (20-30 mesh), 22 g. The column volume of the filled column was determined by measuring the time for THF to fill the column at 1 mL/min flow rate. Column volume=4.3 mL. The zinc was activated by flowing a strong activating solution through the column at 0.5 mL/min for 10 mins. The strong activating solution consists of 1 mL TMSCl (0.67 M) & 0.75 mL chlorobromoethane (0.71 M) in 10 mL THF. After activation, the column was washed with dry THF: 10 mL, 1 mL/min. tert-butyl (R)-3-(iodomethyl)pyrrolidine-1-carboxylate (10 g, 37 mmol) was dissolved in THF (60 mL). The iodide solution was flowed through the activated zinc column at 50° C., flow rate 0.45 mL/min. After reaction: titration with iodine shows a concentration of 0.30 M.

Intermediate 6 (1.2 g, 2.4 mmol) was added with RuPhos Pd G4 (0.051 g, 0.06 mmol) in a sealed vial with a stirring bar in a glove box. Then, a solution of freshly made R-((1-(tert-butoxycarbonyl)-3-yl)methyl)zinc(II) iodide (12 mL, 0.3 M, 3.6 mmol) which was prepared by the above procedure was added. Next, the solution was heated to 50° C. under nitrogen atmosphere during 16 h. The solution was concentrated in vacuo and the residue redissolved in DCM. Next, water was added, followed by aq. Na₄EDTA solution (pH>10). The layers were separated and the water layer was extracted once more with DCM. Organic layers were combined, dried over Na₂SO₄, filtered and evaporated to dryness. The residue was purified by silica gel column chromatography eluting with methanol in dichloromethane from 0% to 10% to give intermediate 201 (1.4 g, 1.5 mmol (55% purity), 63% yield).

### Preparation of Intermediate 202:

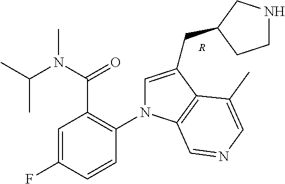

The mixture of intermediate 201 (17 g, 3.09 mmol) in dichloromethane (50 mL), was added the solution 24 mL of chlorine hydride (7 M in ethyl acetate). After stirring at r.t. for 5 h, the reaction mixture was concentrated, and the residue was diluted with DCM and basified with sodium hydroxide aqueous solution (1M) to pH˜10. The layers were separated and the aqueous layer was extracted with DCM three times and the combined organic layer was washed with brine (30 mL), dried over sodium sulfate, filtered and concentrated to afford intermediate 202 (13 g, 31.1 mmol, 94.2% yield) as a yellow solid, which was used in the next step without purification.

Alternatively, intermediate 202 can also be prepared as a 0.2TFA salt by using the following procedure:

Intermediate 201 (5.2 g, 6.95 mmol, 68% pure) is dissolved in DCM (44.5 mL) and TFA (5.3 mL) was added and stirred for 4 h at t. The solution was concentrated in vacuo and coevaporated with toluene. Next, the mixture was washed with 1M NaOH and extracted four times with 10 DCM and EtOAc and Me-THF to obtain the combined organics which were then dried with anhydrous Na₂SO₄, filtered and concentrated in vacuo. The residue was purified via by silica gel column chromatography eluting with methanol (containing 7N NH₃) in dichloromethane from 0% to 10% to give intermediate 202 as a 0.2TFA salt.

Alternatively, intermediate 202 can also be prepared with the following procedure:

Into a 10 L 4-necked round-bottom flask were added 4N HCl in 1,4-dioxane (1.8 L). Then, crude intermediate 201 in THF (3 L) was added dropwise (calculated by 735 g intermediate 201, 1.82 mol, 1.0 equiv) at 0° C. The resulting mixture was stirred for an additional 2 h at 0° C. The resulting mixture was diluted with ethyl acetate (3 L) and water (3 L). The aqueous layer was washed with DCM (10×1 L). The pH of the aqueous layer was adjusted to pH 8 with saturated aqueous Na₂CO₃ solution and extracted with CH₂Cl₂ (4×2 L). The organic layers were dried with Na₂SO₄ and concentrated under vacuum to afford intermediate 202 (389 g, yield 53% over 2 steps) as a light yellow solid.

### Preparation of Intermediate Z:

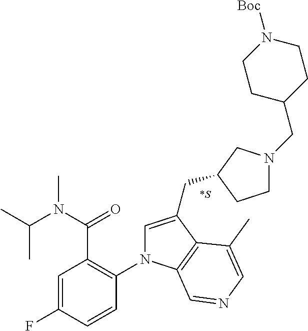

To a solution of intermediate 27 (3.5 g, 95%, 8.14 mmol) in DCM (80 mL) was added tert-butyl 4-formylpiperidine-1-carboxylate (3.66 g, 16.3 mmol) and sodium triacetoxyborohydride (2.58 g, 12.2 mmol). After stirring at room temperature for 6 hours, the reaction mixture was poured into saturated aqueous sodium bicarbonate solution and extracted with dichloromethane (80 mL) twice. The combined organic layers were washed with brine (80 mL), dried over Na₂SO₄, filtered and concentrated under reduced pressure to afford the crude product, which was purified by silica gel column chromatography eluting with methanol in dichloromethane from 0% to 6% to give intermediate Z (4.66 g, 95% purity, 89.8% yield) as a white oil.

### Preparation of Intermediate Y:

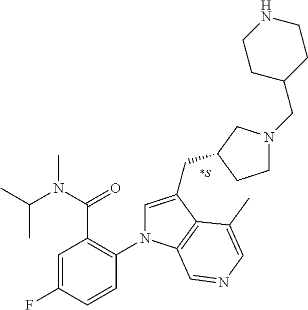

At 0° C., to a solution of intermediate Z (650 mg, 95% purity, 1.02 mmol) in DCM (8 mL) was added hydrogen chloride in ethyl acetate (2.2 mL, 7 M). After stirring at room temperature for 2 hours, the reaction mixture was concentrated and the residue was basified with aqueous sodium hydroxide solution (1M) and extracted with DCM (20 mL) twice. The combined organic layers were washed with brine (20 mL), dried over Na₂SO₄, filtered and concentrated to afford intermediate Y, which was used in the next step without purification.

### Preparation of Compound 51:

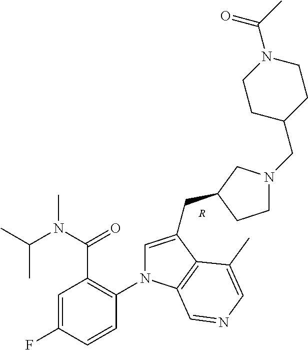

At 0° C., to a solution of intermediate Y (1.04 g, 95% purity, 1.95 mmol) in DCM (10 mL) was added acetyl chloride (160 mg, 2.05 mmol) and triethylamine (592 mg, 5.85 mmol). After stirring at room temperature for 2 hours, the resulting mixture was poured into water and extracted with dichloromethane (20 mL) twice. The combined organic layers were washed with brine (20 mL), dried over Na₂SO₄, filtered and concentrated to afford the crude product, which was purified by prep HPLC (Column: Xbrige C18 150*19 mm*5 um, Mobile Phase A: water (0.1% NH₄HCO₃), Mobile Phase B: acetonitrile, Flow rate: 15 mL/min, gradient condition from 15% B to 60% B). The collected fraction was lyophilized to give Compound 51 (1.25 g, 99.8% purity, 74.9% yield) as a white solid.

Alternatively, Compound 51 can also be prepared with the following procedure:

Intermediate 202 (as 0.2TFA salt) (0.20 g, 0.49 mmol) and 1-acetylpiperidine-4-carbaldehyde (0.097 g, 0.62 mmol) were dissolved in MeOH (5.5 mL). After stirring at ambient temperature for ˜5 min, solid NaCNBH₃ (0.039 g, 0.62 mmol) was added. The resulting mixture was stirred at ambient temperature for ˜2 h, after which sat. aq. NaHCO₃ solution was added. Then, most of the MeOH was evaporated to dryness, and DCM was added. The pH of the water layer was adjusted to pH>10 with 1M aq. NaOH solution. The layers were separated and the water layer was extracted three times more with DCM. The organic layers were combined, dried over Na₂SO₄, filtered and evaporated. The residue was purified by silica gel column chromatography eluting with methanol (+1% 7N NH₃ in MeOH) in dichloromethane from 0% to 10% to give Compound 51 (0.060 g, 0.11 mmol, 35% yield).

Compound 51 (originating from route via intermediate 202; 0.051 g, purity 99.7%, LC/MS method 32) was dissolved in 2-3 drops of isopropylacetate (IPAC), after which the resulting solution was stirred at 45° C. for ˜5 h. Next, the mixture was allowed to stir at ambient temperature for 48 h, after which it was filtered to obtain a white solid material corresponding with Compound 51 in its crystalline free base Form. Melting point (via DSC): Tₒₙₛₑₜ=121.6° C.

Compound 51 ((originating from route via intermediate 202; ˜1 g, 98.7% purity, LC/MS method 33) was dissolved in cyclopentylmethylether (CPME) (3 mL), after which heptane (2 mL) was slowly added, followed by the addition of ˜10 mg of seeding crystals (obtained via previous procedure). Next, 1 mL of heptane was added and the mixture stirred for 20 h, after which the suspension was filtered to give solid material which was dried at 40° C. under vacuum to yield Compound 51 in its crystalline free base Form (96% yield).

Chiral SFC method 1 was employed to match the stereochemistry of Compound 51 obtained through the route employing intermediate Y or intermediate 202; retention time=4.73-4.77 min.

### Preparation of Compound 51a:

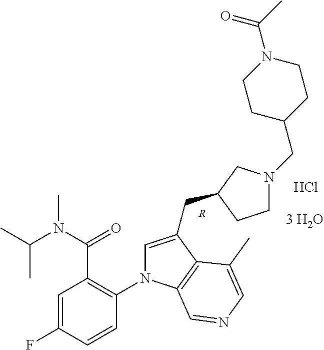

Compound 51 (0.50 g, 0.91 mmol, purity 95.2% (determined by LC/MS method 32)) was dissolved in acetone (0.50 mL) and stirred to give a clear solution. Next, a solution of 1M HCl in acetone was prepared as follows: 1 mL of concentrated aq. HCl solution was added to 11 mL of acetone. Then, a solution of 1M HCl in acetone (0.92 mL, 1 eq.) was added, keeping a solution. The solution was stirred at ambient temperature for ˜30-60 min, after which heptane (5.0 mL) was added. Next, acetone was added (3.0 mL). Vigorous stirring was initiated, and the mixture was stirred overnight. Then, a fine white suspension was obtained, and the suspension was filtered. The solid was rinsed with heptane and dried to give Compound 51a as a mono HCl trihydrate salt (when determined via dynamic vapor sorption analysis around 3 equivalents water) as a white solid (0.48 g, yield 78%). Melting point (via DSC): Tₒₙₛₑₜ=139° C.

Compound 51a was obtained as a variable hydrate with equilibrated water content varying as function of humidity—mainly trihydrate at ambient % relative humidity.

### Pharmacology

It has been found that the compounds of the present invention block the interaction of menin with MLL proteins and oncogenic MLL fusion proteins per se, or can undergo metabolism to a (more) active form in vivo (prodrugs). Therefore the compounds according to the present invention and the pharmaceutical compositions comprising such compounds may be useful for the treatment or prevention, in particular treatment, of diseases such as cancer, including but not limited to leukemia, myelodysplastic syndrome (MDS), and myeloproliferative neoplasms (MPN); and diabetes.

In particular, the compounds according to the present invention and the pharmaceutical compositions thereof may be useful in the treatment or prevention of cancer. According to one embodiment, cancers that may benefit from a treatment with menin/MLL inhibitors of the invention comprise leukemias, lymphomas, myelomas or solid tumor cancers (e.g. prostate cancer, lung cancer, breast cancer, pancreatic cancer, colon cancer, liver cancer, melanoma and glioblastoma, etc.). In some embodiments, the leukemias include acute leukemias, chronic leukemias, myeloid leukemias, myelogeneous leukemias, lymphoblastic leukemias, lymphocytic leukemias, Acute myelogeneous leukemias (AML), Chronic myelogenous leukemias (CML), Acute lymphoblastic leukemias (ALL), Chronic lymphocytic leukemias (CLL), T cell prolymphocytic leukemias (T-PLL), Large granular lymphocytic leukemia, Hairy cell leukemia (HCL), MLL-rearranged leukemias, MLL-PTD leukemias, MLL amplified leukemias, MLL-positive leukemias, leukemias exhibiting HOX/MEIS1 gene expression signatures etc.

In particular, the compounds according to the present invention and the pharmaceutical compositions thereof may be useful in the treatment or prevention of myelodysplastic syndrome (MDS) or myeloproliferative neoplasms (MPN).

In particular, compounds according to the present invention and the pharmaceutical compositions thereof may be useful in the treatment or prevention of leukemias, in particular nucleophosmin (NPM1)-mutated leukemias, e.g. NPM1c.

In particular, compounds according to the present invention and the pharmaceutical compositions thereof may be useful in the treatment or prevention of AML, in particular nucleophosmin (NPM1)-mutated AML (i.e., NPM1^{mut} AML), more in particular abstract NPM1-mutated AML.

In particular, compounds according to the present invention and the pharmaceutical compositions thereof may be useful in the treatment or prevention of MLL-rearranged leukemias, in particular MLL-rearranged AML or ALL.

In particular, compounds according to the present invention and the pharmaceutical compositions thereof may be useful in the treatment or prevention of leukemias with MLL gene alterations, in particular AML or ALL with MLL gene alterations.

In particular, compounds according to the present invention and the pharmaceutical compositions thereof may be suitable for Q.D. dosing (once daily).

In particular, compounds according to the present invention and the pharmaceutical compositions thereof may be useful in the treatment or prevention of hematological cancer in a subject exhibiting NPM1 gene mutations and/or mixed lineage leukemia gene (MLL; MLL1; KMT2A) alterations, mixed lineage leukemia (MLL), MLL-related leukemia, MLL-associated leukemia, MLL-positive leukemia, MLL-induced leukemia, rearranged mixed lineage leukemia, leukemia associated with a MLL rearrangement/alteration or a rearrangement/alteration of the MLL gene, acute leukemia, chronic leukemia, myelodysplastic syndrome (MDS), myeloproliferative neoplasms (MPN), insulin resistance, pre-diabetes, diabetes, or risk of diabetes, hyperglycemia, chromosomal rearrangement on chromosome 11q23, type-1 diabetes, type-2 diabetes; promoting proliferation of a pancreatic cell, where pancreatic cell is an islet cell, beta cell, the beta cell proliferation is evidenced by an increase in beta cell production or insulin production; and for inhibiting a menin-MLL interaction, where the MLL fusion protein target gene is HOX or MEIS1 in human.

Hence, the invention relates to compounds according to the present invention, for use as a medicament.

The invention also relates to the use of a compound according to the present invention, or a pharmaceutical composition according to the invention, for the manufacture of a medicament.

The present invention also relates to a compound according to the present invention, or a pharmaceutical composition according to the invention, for use in the treatment, prevention, amelioration, control or reduction of the risk of disorders associated with the interaction of menin with MLL proteins and oncogenic MLL fusion proteins in a mammal, including a human, the treatment or prevention of which is affected or facilitated by blocking the interaction of menin with MLL proteins and oncogenic MLL fusion proteins.

Also, the present invention relates to the use of a compound according to the present invention, or a pharmaceutical composition according to the invention, for the manufacture of a medicament for treating, preventing, ameliorating, controlling or reducing the risk of disorders associated with the interaction of menin with MLL proteins and oncogenic MLL fusion proteins in a mammal, including a human, the treatment or prevention of which is affected or facilitated by blocking the interaction of menin with MLL proteins and oncogenic MLL fusion proteins.

The invention also relates to a compound according to the present invention, for use in the treatment or prevention of any one of the diseases mentioned hereinbefore.

The invention also relates to a compound according to the present invention, for use in treating or preventing any one of the diseases mentioned hereinbefore.

The invention also relates to the use of a compound according to the present invention, for the manufacture of a medicament for the treatment or prevention of any one of the disease conditions mentioned hereinbefore.

The compounds of the present invention can be administered to mammals, preferably humans, for the treatment or prevention of any one of the diseases mentioned hereinbefore.

In view of the utility of the compounds according to the present invention, there is provided a method of treating warm-blooded animals, including humans, suffering from any one of the diseases mentioned hereinbefore.

Said method comprises the administration, i.e. the systemic or topical administration, of a therapeutically effective amount of a compound according to the present invention, to warm-blooded animals, including humans.

Therefore, the invention also relates to a method for the treatment or prevention of any one of the diseases mentioned hereinbefore comprising administering a therapeutically effective amount of compound according to the invention to a patient in need thereof.

One skilled in the art will recognize that a therapeutically effective amount of the compounds of the present invention is the amount sufficient to have therapeutic activity and that this amount varies inter alias, depending on the type of disease, the concentration of the compound in the therapeutic formulation, and the condition of the patient. An effective therapeutic daily amount would be from about 0.005 mg/kg to 100 mg/kg. The amount of a compound according to the present invention, also referred to herein as the active ingredient, which is required to achieve a therapeutically effect may vary on case-by-case basis, for example with the particular compound, the route of administration, the age and condition of the recipient, and the particular disorder or disease being treated. A method of treatment may also include administering the active ingredient on a regimen of between one and four intakes per day. In these methods of treatment the compounds according to the invention are preferably formulated prior to administration.

The present invention also provides compositions for preventing or treating the disorders referred to herein. Said compositions comprising a therapeutically effective amount of a compound according to the present invention, and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions may be prepared by any methods well known in the art of pharmacy.

The compounds of the present invention may be administered alone or in combination with one or more additional therapeutic agents. Combination therapy includes administration of a single pharmaceutical dosage formulation which contains a compound according to the present invention and one or more additional therapeutic agents, as well as administration of the compound according to the present invention and each additional therapeutic agent in its own separate pharmaceutical dosage formulation.

Therefore, an embodiment of the present invention relates to a product containing as first active ingredient a compound according to the invention and as further active ingredient one or more anticancer agent, as a combined preparation for simultaneous, separate or sequential use in the treatment of patients suffering from cancer.

The one or more other medicinal agents and the compound according to the present invention may be administered simultaneously (e.g. in separate or unitary compositions) or sequentially in either order. In the latter case, the two or more compounds will be administered within a period and in an amount and manner that is sufficient to ensure that an advantageous or synergistic effect is achieved. It will be appreciated that the preferred method and order of administration and the respective dosage amounts and regimes for each component of the combination will depend on the particular other medicinal agent and compound of the present invention being administered, their route of administration, the particular condition, in particular tumour, being treated and the particular host being treated.

### LCMS (Liquid Chromatography/Mass Spectrometry)

### General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl), the reported value is the one obtained for the lowest isotope mass. All results were obtained with experimental uncertainties that are commonly associated with the method used.

Hereinafter, “SQD” means Single Quadrupole Detector, “RT” room temperature, “BEH” bridged ethylsiloxane/silica hybrid, “HSS” High Strength Silica, “DAD” Diode Array Detector.

TABLE 1a
LCMS Method codes (Flow expressed in mL/min; column temperature (T) in °C.;
Run time in minutes). “TFA” means trifluoroacetic acid; “FA” means formic acid

| Method code | Instrument | Column | Mobile phase | Gradient | $\frac{\begin{matrix}Flow \\ ( ml / min )\end{matrix}}{\begin{matrix}Column \\ T ( ° C . )\end{matrix}}$ | Run time (min) |
|---|---|---|---|---|---|---|
| 8 | Agilent Technologies 1200 Series, G6110A | ZORBAX SB-C8, 3.5 um 4.6 * 150 mm | A: 0.05% TFA; B: CH₃CN | 90% A for 3.00 min, to 5% A in 8.00 min, held for 3.60 min, back to 90% A in 0.10 min, held for 0.30 min. | $\frac{1.5}{40}$ | 15 |
| 14 | Agilent: 1260 Infinity and 6120 Quadrupole LC/MS | Waters: Sunfire C18 (2.5 μm, 3.0 × 30 mm) | A: 0.1% FA solution in water; B: CH₃CN | Gradient start from 5% of B increase to 95% within 2.5 min and keep at 95% till 3.5 min | $\frac{1.2}{50}$ | 3.5 |

TABLE 1b
LCMS and melting point data.

| Co. No. | Rt | [M + 1]⁺ | LCMS method |
|---|---|---|---|
| 51 | 7.28 | 548.2 | 8 |
| Intermediate Z | 1.69 | 606.84 | 14 |

Co. No. means compound number; Rₜ means retention time in min.

### Analytical SFC

### General Procedure for SFC Methods

The SFC measurement was performed using an Analytical Supercritical fluid chromatography (SFC) system composed by a binary pump for delivering carbon dioxide (CO₂) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

TABLE 1c
Analytical SFC Methods (Flow expressed in mL/min; column temperature (T)
in °C.; Run time in minutes, Backpressure (BPR) in bars or pound-force per square inch (psi).
“ACN” means acetonitrile; “MeOH” means methanol; “EtOH” means ethanol; “iPrNH₂”
means isopropylamine. All other abbreviations used in the table below are as defined before

| Method code | Column | Mobile phase | Gradient | $\frac{\begin{matrix}Flow - \\ ( ml / min )\end{matrix}}{\begin{matrix}Column T \\ ( ° C . )\end{matrix}}$ | $\frac{\begin{matrix}Run Time \\ ( min )\end{matrix}}{BPR ( bar )}$ |
|---|---|---|---|---|---|
| 1 | Daicel Chiralpak ® AD3 column (3.0 μm, 150 × 4.6 mm) | A: supercritical CO₂ B: iPrOH +0.2% iPrNH₂ | 10%-50% B in 6 min, hold 3.5 min | $\frac{2.5}{40}$ | $\frac{9.5}{130}$ |
| 2 | Daicel Chiralpak ® IG3 column (3.0 μm, 150 × 4.6 mm) | A: supercritical CO₂ B: EtOH +0.2% iPrNH₂ | 10%-50% B in 6 min, hold 3.5 min | $\frac{2.5}{40}$ | $\frac{9.5}{130}$ |

### NMR:

### NMR—Methods

Some NMR experiments were carried out using a Bruker Avarnce II11400 spectrometer at ambient temperature (298.6 K), using internal deuterium lock and equipped with BBO 400 MHz S1 5 mm probe head with z gradients and operating at 400 MHz for the proton and 100 MHz for carbon. Chemical shifts (d) are reported in parts per million (ppm). J values are expressed in Hz.

Some NMR experiments were carried out using a Varian 400-MR spectrometer at ambient temperature (298.6 K), using internal deuterium lock and equipped with Varian 400 4NUC PFG probe head with z gradients and operating at 400 MHz for the proton and 100 MHz for carbon. Chemical shifts (d) are reported in parts per million (ppm). J values are expressed in Hz.

Some NMR experiments were carried out using a Varian 400-VNMRS spectrometer at ambient temperature (298.6 K), using internal deuterium lock and equipped with Varian 400 ASW PFG probe head with z gradients and operating at 400 MHz for the proton and 100 MHz for carbon. Chemical shifts (d) are reported in parts per million (ppm). J values are expressed in Hz.

| Compound | |
|---|---|
| number | NMR data |
| Compound 51 | ¹H NMR (400 MHz, DMSO-d6): δ 8.43-8.34 (m, 1H), 7.94-7.92 (m, 1H), |
| | 7.67-7.62 (m, 1H), 7.50-7.42 (m, 2H), 7.28-7.22 (m, 1H), 4.42-4.39 (m, |
| | 0.43H), 4.34-4.31 (m, 1H), 3.78-3.74 (m, 1H), 3.51-3.46 (m, 0.53H), 3.00- |
| | 2.84 (m, 3H), 2.71-2.55 (m, 7H), 2.48-2.34 (m, 4H), 2.28-2.14 (m, 3H), |
| | 2.00-1.90 (m, 4H), 1.75-1.65 (m, 3H), 1.50-1.44 (m, 1H), 1.06-0.84 (m, |
| | 5H), 0.48-0.17 (m, 3H). |
| Compound 51a | ¹H NMR (400 MHz, DMSO-d₆, 27° C.) δ ppm 0.18-0.53 (m, 3 H), 0.89- |
| | 1.29 (m, 5 H), 1.68-1.89 (m, 3 H), 1.98 (d, J = 1.3 Hz, 4 H), 2.07-2.29 |
| | (m, 1 H), 2.37 (s, 1 H), 2.57 (s, 2 H), 2.63 (s, 3 H), 2.94-3.15 (m, 6 H), |
| | 3.48-3.54 (m, 0.5 H), 3.56-3.75 (m, 2 H), 3.76-3.86 (m, 1 H), 4.35 (br |
| | d, J = 14.3 Hz, 2 H), 4.38-4.42 (m, 0.5 H), 7.32-7.42 (m, 1 H), 7.44- |
| | 7.54 (m, 2 H), 7.62-7.71 (m, 1 H), 7.97 (d, J = 5.5 Hz, 1 H), 8.34-8.47 |
| | (m, 1 H), 10.01-10.48 (m, 1 H). |

### DSC

For a number of compounds, melting points (MP) were determined with a TA Instrument (Discovery DSC 250 or a DSC 2500). Melting points were measured with a temperature gradient of 10° C./minute. Maximum temperature was 300° C. Values are melting peak onset values.

### XPRD

### Compound 51 as a Crystalline Free Base Form

Compound 51 as a crystalline free base Form may be characterized by an X-ray powder diffraction pattern.

X-ray powder diffraction (XRPD) analysis was carried out on a PANalytical Empyrean diffractometer. The compound was loaded onto a zero-background silicon wafer sample holder by gently pressing the powder sample onto the flat surface.

Samples were run on XRPD using the method below:
Radiation: Cu K-Alpha (λ=1.5418 Å)
Tube voltage/current: 45 kV/40 mA
Divergence slit: ⅛°
Geometry: Bragg-Brentano
Scan mode: Continuous Scan
Scan Range: 3-40° 2θ
Step size: 0.013° 2θ
Scan speed: 20.4 s/step
Rotation: On
Detector: PIXcel^{ID} One skilled in the art will recognize that diffraction patterns and peak positions are typically substantially independent of the diffractometer used and whether a specific calibration method is utilized. Typically, the peak positions may differ by about ±0.2° 20, or less. The intensities (and relative intensities) of each specific diffraction peak may also vary as a function of various factors, including but not limited to particle size, orientation, sample purity, etc.

The X-ray powder diffraction pattern comprises peaks at 9.3, 12.6, 15.7, 21.9 and 22.5° 2θ±0.2° 2θ. The X-ray powder diffraction pattern may further comprise at least one peak selected from 8.1, 11.6, 13.2, 16.8, 18.5, 18.7, 19.2, 19.9, 20.5° 2θ±0.2° 2θ.

Compound 51 as a crystalline free base Form may further be characterized by an X-ray powder diffraction pattern having four, five, six, seven, eight, nine or more peaks selected from those peaks identified in Table 2a.

Compound 51 as a crystalline free base Form may further be characterized by an X-ray powder diffraction pattern comprising those peaks identified in Table 2a, wherein the relative intensity of the peaks is greater than about 2%, preferably greater than about 5%, more preferably greater than about 10%, more preferably greater than about 15%. However, a skilled person will realize that the relative intensity of the peaks may vary between different samples and different measurements on the same sample.

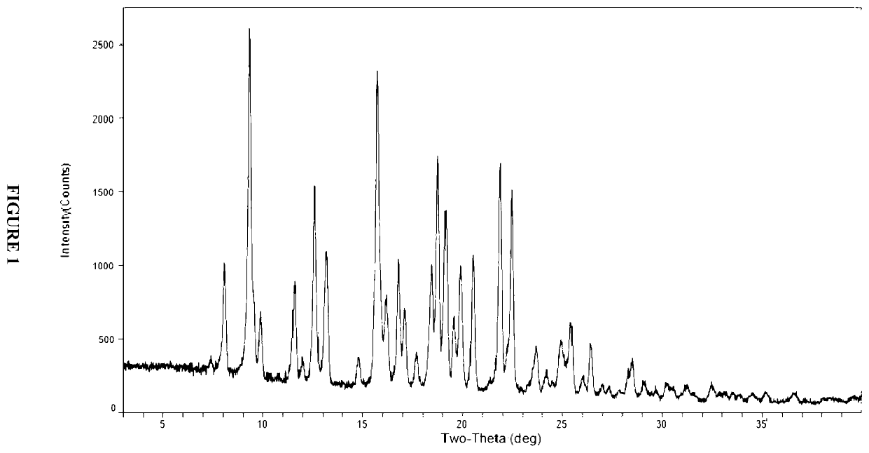

Compound 51 as a crystalline free base Form may further be characterized by an X-ray powder diffraction pattern substantially as depicted in FIG. 1.

TABLE 2a
provides peak listing and relative intensity for the
XPRD of Compound 51 as a crystalline free base Form:

| | Pos. | Rel. Int. |
|---|---|---|
| No. | (°2θ) | (%) |
| 1 | 7.411 | 4.6 |
| 2 | 8.056 | 31.4 |
| 3 | 9.304 | 100 |
| 4 | 9.893 | 18.2 |
| 5 | 11.574 | 28.1 |
| 6 | 11.969 | 6.4 |
| 7 | 12.598 | 55.9 |
| 8 | 13.163 | 37.6 |
| 9 | 14.804 | 7.9 |
| 10 | 15.723 | 89.6 |
| 11 | 16.195 | 24.4 |
| 12 | 16.762 | 34.5 |
| 13 | 17.076 | 21.2 |
| 14 | 17.694 | 9.5 |
| 15 | 18.454 | 33.2 |
| 16 | 18.744 | 63.6 |
| 17 | 19.15 | 48.8 |
| 18 | 19.57 | 16.4 |
| 19 | 19.912 | 32.2 |
| 20 | 20.503 | 38.2 |
| 21 | 21.881 | 65.4 |
| 22 | 22.485 | 57.1 |
| 23 | 23.693 | 13 |
| 24 | 24.205 | 5.6 |
| 25 | 24.915 | 15.2 |
| 26 | 25.401 | 19.6 |
| 27 | 26.068 | 5.3 |
| 28 | 26.400 | 14.5 |
| 29 | 28.276 | 8 |
| 30 | 28.499 | 11 |

### Compound 51a Crystalline HCl Salt Form (Mono HCl Trihydrate Salt)

Compound 51a (Crystalline HCl salt Form—mono HCl trihydrate salt—Compound 51a was obtained as a variable hydrate with equilibrated water content varying as function of humidity mainly trihydrate at ambient % relative humidity) may be characterized by an X-ray powder diffraction pattern.

X-ray powder diffraction (XRPD) analysis was carried out on a PANalytical Empyrean diffractometer. The compound was loaded onto a zero-background silicon wafer sample holder by gently pressing the powder sample onto the flat surface.

Samples were run on XRPD using the method below:
Radiation: Cu K-Alpha (k=1.5418 Å)
Tube voltage/current: 45 kV/40 mA
Divergence slit: ⅛°
Geometry: Bragg-Brentano
Scan mode: Continuous Scan
Scan Range: 3-40° 2θ
Step size: 0.013° 2θ
Scan speed: 20.4 s/step
Rotation: On
Detector: PIXcel^{ID} One skilled in the art will recognize that diffraction patterns and peak positions are typically substantially independent of the diffractometer used and whether a specific calibration method is utilized. Typically, the peak positions may differ by about ±0.2° 20, or less. The intensities (and relative intensities) of each specific diffraction peak may also vary as a function of various factors, including but not limited to particle size, orientation, sample purity, etc.

The X-ray powder diffraction pattern comprises peaks at 5.2, 13.2, 14.1, 18.8 and 20.3° 2θ±0.2° 2θ. The X-ray powder diffraction pattern may further comprise at least one peak selected from 9.7, 10.0, 15.4, 15.8, 18.3, 21.3, 24.3° 2θ±0.2° 2θ.

Compound 51a may further be characterized by an X-ray powder diffraction pattern having four, five, six, seven, eight, nine or more peaks selected from those peaks identified in Table 2b.

Compound 51a may further be characterized by an X-ray powder diffraction pattern comprising those peaks identified in Table 2b, wherein the relative intensity of the peaks is greater than about 2%, preferably greater than about 5%, more preferably greater than about 10%, more preferably greater than about 15%. However, a skilled person will realize that the relative intensity of the peaks may vary between different samples and different measurements on the same sample.

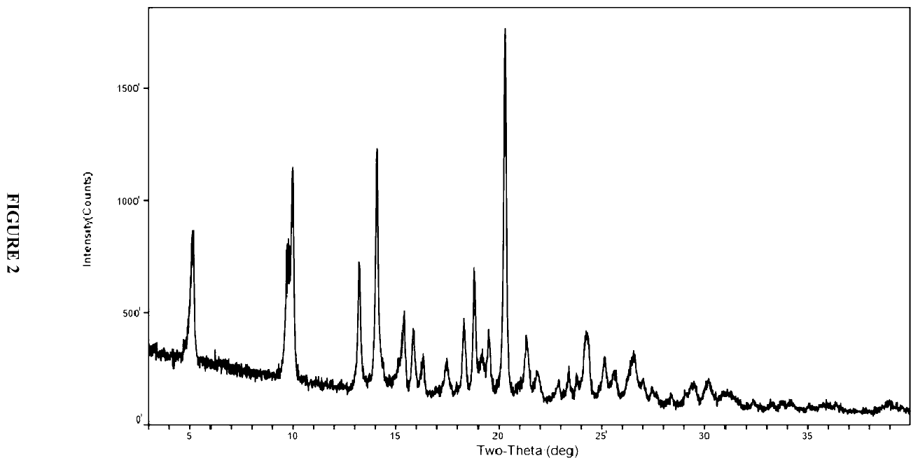

Compound 51a may further be characterized by an X-ray powder diffraction pattern substantially as depicted in FIG. 2.

TABLE 2b
provides peak listing and relative intensity
for the XPRD of Compound 51a.

| | Pos. | Rel. Int. |
|---|---|---|
| No. | (°2θ) | (%) |
| 1 | 5.151 | 36 |
| 2 | 9.749 | 37.2 |
| 3 | 9.984 | 58.9 |
| 4 | 13.217 | 34 |
| 5 | 14.095 | 64.4 |
| 6 | 15.393 | 20.4 |
| 7 | 15.842 | 16.4 |
| 8 | 16.315 | 10.4 |
| 9 | 17.471 | 10.1 |
| 10 | 18.296 | 19.4 |
| 11 | 18.810 | 34.3 |
| 12 | 19.19 | 10.8 |
| 13 | 19.505 | 16.6 |
| 14 | 20.305 | 100 |
| 15 | 21.331 | 16.6 |
| 16 | 21.855 | 6.8 |
| 17 | 22.905 | 4.5 |
| 18 | 23.419 | 8.2 |
| 19 | 24.310 | 17.4 |
| 20 | 25.136 | 10.6 |
| 21 | 25.595 | 7.1 |
| 22 | 26.529 | 12.9 |
| 23 | 29.496 | 4.5 |
| 24 | 30.179 | 6.1 |

### Dynamic Vapor Sorption (DVS)

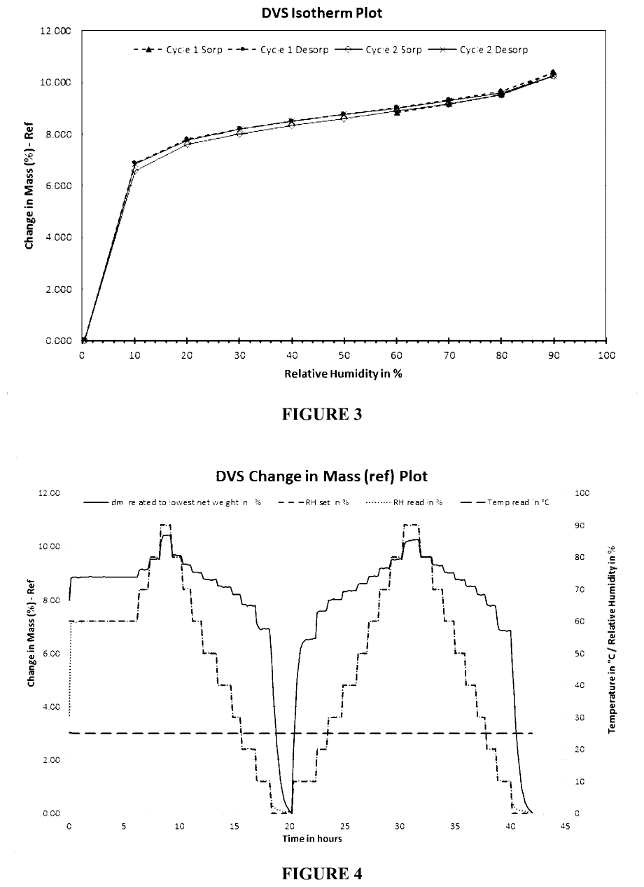

The moisture sorption analysis (DVS) was performed using a ProUmid GmbH & Co. KG Vsorp Enhanced dynamic vapor sorption apparatus. Results are shown in FIG. 3 and FIG. 4. The moisture profile was evaluated by monitoring vapor adsorption/desorption over the range of 0 to 90% relative humidity at 25° C. The sample weight equilibrium criteria were set at ≤0.010% change in 45 min with minimum and maximum time of acclimation at 50 min and 120 min, respectively. The moisture profile consisted of 2 cycles of vapor adsorption/desorption.

The DVS change in mass plot of crystalline HCl salt Form (Compound 51a) shows that the crystalline form is hygroscopic with the water content varying with relative humidity and dehydrates rapidly at below 10% RH (relative humidity) to complete dehydrated state at 0% RH. In the humidity range of 20-90% RH, the crystalline form adsorbs and desorbs moisture slowly and reversibly up to 2.5% by mass on average. Based on DVS, the crystalline HCl salt Form, at equilibrium, can contain around 3 equivalents of water (8.5-9.5% total moisture mass) at common ambient RH of 40% to 75%. The XRPD pattern of the fraction obtained after the DVS test was comparable to the starting material. No indication of a solid-state form change was observed.

### Pharmacological Part

### 1) Menin/MLL Homogenous Time-Resolved Fluorescence (HTRF) Assay

To an untreated, white 384-well microtiter plate was added 40 nL 200× test compound in DMSO and 4 μL 2× terbium chelate-labeled menin (vide infra for preparation) in assay buffer (40 mM Tris·HCl, pH 7.5, 50 mM NaCl, 1 mM DTT (dithiothreitol) and 0.05% Pluronic F-127). After incubation of test compound and terbium chelate-labeled menin for 30 min at ambient temperature, 4 μL 2×FITC-MBM1 peptide (FITC-β-alanine-SARWRFPARPGT-NH₂) (“FITC” means fluorescein isothiocyanate) in assay buffer was added, the microtiter plate centrifuged at 1000 rpm for 1 min and the assay mixtures incubated for 15 min at ambient temperature. The relative amount of menin-FITC-MBM1 complex present in an assay mixture is determined by measuring the homogenous time-resolved fluorescence (HTRF) of the terbium/FITC donor/acceptor fluorphore pair using an EnVision microplate reader (ex. 337 nm/terbium em. 490 nm/FITC em. 520 nm) at ambient temperature. The degree of fluorescence resonance energy transfer (the HTRF value) is expressed as the ratio of the fluorescence emission intensities of the FITC and terbium fluorophores (F^{em} 520 nm/F^{em} 490 nm). The final concentrations of reagents in the binding assay are 200 pM terbium chelate-labeled menin, 75 nM FITC-MBM1 peptide and 0.5% DMSO in assay buffer. Dose-response titrations of test compounds are conducted using an 11 point, four-fold serial dilution scheme, starting typically at 10 μM.

Compound potencies were determined by first calculating % inhibition at each compound concentration according to equation 1:

$$\% inhibition = ( ( HC - LC ) - ( {HTRF}^{compound} - LC ) ) / ( HC - LC ) ) * 100 \qquad ( Eqn 1 )$$

Where LC and HC are the HTRF values of the assay in the presence or absence of a saturating concentration of a compound that competes with FITC-MBM1 for binding to menin, and HTRF^{compound} is the measured HTRF value in the presence of the test compound. HC and LC HTRF values represent an average of at least 10 replicates per plate. For each test compound, % inhibition values were plotted vs. the logarithm of the test compound concentration, and the IC₅₀ value derived from fitting these data to equation 2:

% inhibition=Bottom+(Top−Bottom)/(1+10\hat{ }((log IC₅₀−log[cmpd])*h)) (Eqn 2)

Where Bottom and Top are the lower and upper asymptotes of the dose-response curve, respectively, IC₅₀ is the concentration of compound that yields 50% inhibition of signal and h is the Hill coefficient.

Preparation of Terbium cryptate labeling of Menin: Menin (a.a 1-610-6×his tag, 2.3 mg/mL in 20 mM Hepes (2-[4-(2-Hydroxyethyl)-1-piperazinyl]ethane sulfonic acid), 80 mM NaCl, 5 mM DTT (Dithiothreitol), pH 7.5) was labeled with terbium cryptate as follows. 200 μg of Menin was buffer exchanged into 1× Hepes buffer. 6.67 μM Menin was incubated with 8-fold molar excess NHS (N-hydroxysuccinimide)-terbium cryptate for 40 minutes at room temperature. Half of the labeled protein was purified away from free label by running the reaction over a NAP5 column with elution buffer (0.1M Hepes, pH 7+0.1% BSA (bovine serum albumin)). The other half was eluted with 0.1M phosphate buffered saline (PBS), pH7. 400 μl of eluent was collected for each, aliquoted and frozen at −80° C. The final concentration of terbium-labeled Menin protein was 115 μg/mL in Hepes buffer and 85 μg/mL in PBS buffer, respectively.

MENIN Protein Sequence (SEQ ID NO: 1):
MGLKAAQKTLFPLRSIDDVVRLFAAELGREEPDLVLLSLVLGFVEHFLA
VNRVIPTNVPELTFQPSPAPDPPGGLTYFPVADLSIIAALYARFTAQIR
GAVDLSLYPREGGVSSRELVKKVSDVIWNSLSRSYFKDRAHIQSLFSFI
TGTKLDSSGVAFAVVGACQALGLRDVHLALSEDHAWVVFGPNGEQTAEV
TWHGKGNEDRRGQTVNAGVAERSWLYLKGSYMRCDRKMEVAFMVCAINP
SIDLHTDSLELLQLQQKLLWLLYDLGHLERYPMALGNLADLEELEPTPG
RPDPLTLYHKGIASAKTYYRDEHIYPYMYLAGYHCRNRNVREALQAWAD
TATVIQDYNYCREDEEIYKEFFEVANDVIPNLLKEAASLLEAGEERPGE
QSQGTQSQGSALQDPECFAHLLRFYDGICKWEEGSPTPVLHVGWATFLV
QSLGRFEGQVRQKVRIVSREAEAAEAEEPWGEEAREGRRRGPRRESKPE
EPPPPKKPALDKGLGTGQGAVSGPPRKPPGTVAGTARGPEGGSTAQVPA
PAASPPPEGPVLTFQSEKMKGMKELLVATKINSSAIKLQLTAQSQVQMK
KQKVSTPSDYTLSFLKRQRKGLHHHHHH

### 2a) Proliferation Assay

The anti-proliferative effect of menin/MLL protein/protein interaction inhibitor test compounds was assessed in human leukemia cell lines. The cell line MOLM14 harbors a MLL translocation and expresses the MLL fusion protein MLL-AF9, respectively, as well as the wildtype protein from the second allele. OCI-AML3 cells that carry the NPM1c gene mutation were also tested. MLL-rearranged cell lines (e.g. MOLM14) and NPM1c mutated cell lines exhibit stem cell-like HOXA/MEIS1 gene expression signatures. KO-52 was used as a control cell line containing two MLL (KMT2A) wildtype alleles in order to exclude compounds that display general cytotoxic effects.

MOLM14 cells were cultured in RPMI-1640 (Sigma Aldrich) supplemented with 10% heat-inactivated fetal bovine serum (HyClone), 2 mM L-glutamine (Sigma Aldrich) and 50 μg/ml gentamycin (Gibco). KO-52 and OCI-AML3 cell lines were propagated in alpha-MEM (Sigma Aldrich) supplemented with 20% heat-inactivated fetal bovine serum (HyClone), 2 mM L-glutamine (Sigma Aldrich) and 50 μg/ml gentamycin (Gibco). Cells were kept at 0.3-2.5 million cells per ml during culturing and passage numbers did not exceed 20.

In order to assess the anti-proliferative effects, 200 MOLM14 cells, 200 OCI-AML3 cells or 300 KO-52 cells were seeded in 200 μl media per well in 96-well round bottom, ultra-low attachment plates (Costar, catalogue number 7007). Cell seeding numbers were chosen based on growth curves to ensure linear growth throughout the experiment. Test compounds were added at different concentrations and the DMSO content was normalized to 0.3%. Cells were incubated for 8 days at 37° C. and 5% C02. Spheroid like growth was measured in real-time by live-cell imaging (IncuCyteZOOM, Essenbio, 4× objective) acquiring images at day 8. Confluence (%) as a measure of spheroid size was determined using an integrated analysis tool.

In order to determine the effect of the test compounds over time, the confluence in each well as a measure of spheroid size, was calculated. Confluence of the highest dose of a reference compound was used as baseline for the LC (Low control) and the confluence of DMSO treated cells was used as 0% cytotoxicity (High Control, HC).

Absolute IC₅₀ values were calculated as percent change in confluence as follows:

LC=Low Control: cells treated with e.g. 1 μM of the cytotoxic agent staurosporin, or e.g. cells treated with a high concentration of an alternative reference compound

$$HC = High Control : Mean confluence ( \% ) ( DMSO treated cells ) \\ \% Effect = 100 - ( 100 * ( Sample - LC ) / ( HC - LC ) )$$

GraphPad Prism (version 7.00) was used to calculate the IC₅₀. Dose-response equation was used for the plot of % Effect vs Log 10 compound concentration with a variable slope and fixing the maximum to 100% and the minimum to 0%.

2b) MEIS1 mRNA Expression Assay

MEIS1 mRNA expression upon treatment of compound was examined by Quantigene Singleplex assay (Thermo Fisher Scientific). This technology allows for direct quantification of mRNA targets using probes hybridizing to defined target sequences of interest and the signal is detected using a Multimode plate reader Envision (PerkinElmer). The MOLM14 cell line was used for this experiment. Cells were plated in 96-well plates at 3,750 cells/well in the presence of increasing concentrations of compounds. After incubation of 48 hours with compounds, cells were lysed in lysis buffer and incubated for 45 minutes at 55° C. Cell lysates were mixed with human MEIS1 specific capture probe or human RPL28 (Ribosomal Protein L28) specific probe as a normalization control, as well as blocking probes. Cell lysates were then transferred to the custom assay hybridization plate (Thermo Fisher Scientific) and incubated for 18 to 22 hours at 55° C. Subsequently, plates were washed to remove unbound materials followed by sequential addition of preamplifiers, amplifiers, and label probe. Signals (=gene counts) were measured with a Multimode plate reader Envision. IC₅₀s were calculated by dose-response modelling using appropriate software. For all non-housekeeper genes response equal counts corrected for background and relative expression. For each sample, each test gene signal (background subtracted) was divided by the normalization gene signal (RPL28: background subtracted). Fold changes were calculated by dividing the normalized values for the treated samples by the normalized values for the DMSO treated sample. Fold changes of each target gene were used for the calculation of IC₅₀s.

TABLE 3
Biological data - HTRF assay, proliferation
assay, and MEISI mRNA expression assay

| | HTRF- | | spheroid | spheroid | spheroid |
|---|---|---|---|---|---|
| | 30 min | | assay_{—} | assay_OCI- | assay_{—} |
| | incubation | MEIS1 | MOLM14 | AML3 | KO-52 |
| Compound | IC₅₀ | IC₅₀ | IC₅₀ | IC₅₀ | IC₅₀ |
| Number | (μM) | (μM) | (μM) | (μM) | (μM) |
| 51 | 0.000042 | 0.011 | 0.008 | 0.024 | 1.9 |
| 51a | 0.000024 | 0.011 | 0.013 | 0.070 | 1.2 |

1. A pharmaceutical composition comprising a therapeutically effective amount of 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide as a crystalline free base Form, and a pharmaceutically acceptable carrier or excipient.

2. The pharmaceutical composition according to claim 1, wherein the crystalline free base Form is characterized by an X-ray diffraction pattern comprising peaks at 9.3, 12.6, 15.7, 21.9 and 22.5° 2θ±0.2° 2θ.

3. A pharmaceutical composition comprising a therapeutically effective amount of a 2-[3-[[(3R)-1-[(1-acetyl-4-piperidyl)methyl]pyrrolidin-3-yl]methyl]-4-methyl-pyrrolo[2,3-c]pyridin-1-yl]-5-fluoro-N-isopropyl-benzamide as a crystalline HCl salt Form, and a pharmaceutically acceptable carrier or excipient.

4. The pharmaceutical composition according to claim 3, wherein the HCl salt Form is a mono HCl trihydrate.

5. The pharmaceutical composition according to claim 3, wherein the crystalline HCl salt Form is characterized by an X-ray diffraction pattern comprising peaks at 5.2, 13.2, 14.1, 18.8 and 20.3° 2θ±0.2° 2θ.

6. A method of treating cancer comprising administering to a subject in need thereof a therapeutically effective amount of a compound of claim 1, or a pharmaceutical composition comprising the compound.

7. A method of treating leukemia, myelodysplastic syndrome (MDS), and myeloproliferative neoplasms (MPN) comprising administering to a subject in need thereof a therapeutically effective amount of a compound of claim 1, or a pharmaceutical composition comprising the compound.

8. The method of claim 7 wherein the leukemia is a nucleophosmin (NPM1)-mutated leukemia.

9. The method of claim 6, wherein cancer is selected from leukemias, lymphomas, myelomas or solid tumor cancers.

10. The method of claim 7, wherein the leukemia is selected from acute leukemias, chronic leukemias, myeloid leukemias, myelogeneous leukemias, lymphoblastic leukemias, lymphocytic leukemias, Acute myelogeneous leukemias (AML), Chronic myelogenous leukemias (CML), Acute lymphoblastic leukemias (ALL), Chronic lymphocytic leukemias (CLL), T cell prolymphocytic leukemias (T-PLL), Large granular lymphocytic leukemia, Hairy cell leukemia (HCL), MLL-rearranged leukemias, MLL-PTD leukemias, MLL amplified leukemias, MLL-positive leukemias, and leukemias exhibiting HOX/MEIS1 gene expression signatures.

11. The method of claim 6, comprising administering the pharmaceutical composition to the subject.

12. The method of claim 9, wherein the solid tumor cancers are selected from prostate cancer, lung cancer, breast cancer, pancreatic cancer, colon cancer, liver cancer, melanoma and glioblastoma.

13. The method of claim 7, comprising administering the pharmaceutical composition to the subject.

14. The method of claim 9, wherein the leukemias are selected from acute leukemias, chronic leukemias, myeloid leukemias, myelogeneous leukemias, lymphoblastic leukemias, lymphocytic leukemias, Acute myelogeneous leukemias (AML), Chronic myelogenous leukemias (CML), Acute lymphoblastic leukemias (ALL), Chronic lymphocytic leukemias (CLL), T cell prolymphocytic leukemias (T-PLL), Large granular lymphocytic leukemia, Hairy cell leukemia (HCL), MLL-rearranged leukemias, MLL-PTD leukemias, MLL amplified leukemias, MLL-positive leukemias, and leukemias exhibiting HOX/MEIS1 gene expression signatures.


SEQUENCE LISTING (1 sequence)

SEQ ID NO: 1 - protein, 616 aa, synthetic construct
  source 1..616: note = MENIN protein sequence with His tag
MGLKAAQKTLFPLRSIDDVVRLFAAELGREEPDLVLLSLVLGFVEHFLAVNRVIPTNVPELTFQPSPAPDPPGGLTYFPVADLSIIAALYARFTAQIRGAVDLSLYPREGGVSSRELVKKVSDVIWNSLSRSYFKDRAHIQSLFSFITGTKLDSSGVAFAVVGACQALGLRDVHLALSEDHAWVVFGPNGEQTAEVTWHGKGNEDRRGQTVNAGVAERSWLYLKGSYMRCDRKMEVAFMVCAINPSIDLHTDSLELLQLQQKLLWLLYDLGHLERYPMALGNLADLEELEPTPGRPDPLTLYHKGIASAKTYYRDEHIYPYMYLAGYHCRNRNVREALQAWADTATVIQDYNYCREDEEIYKEFFEVANDVIPNLLKEAASLLEAGEERPGEQSQGTQSQGSALQDPECFAHLLRFYDGICKWEEGSPTPVLHVGWATFLVQSLGRFEGQVRQKVRIVSREAEAAEAEEPWGEEAREGRRRGPRRESKPEEPPPPKKPALDKGLGTGQGAVSGPPRKPPGTVAGTARGPEGGSTAQVPAPAASPPPEGPVLTFQSEKMKGMKELLVATKINSSAIKLQLTAQSQVQMKKQKVSTPSDYTLSFLKRQRKGLHHHHHH